(19) United States
(12) Patent Application Publication
(10) Pub. No.: US 2007/0083239 A1
(43) Pub. Date: Apr. 12, 2007

(54) Methods and apparatus for inducing, monitoring and controlling renal neuromodulation

(76) Inventors: Denise Demarais, Los Gatos, CA (US); Nicolas Zadno, Fremont, CA (US)

Correspondence Address: PERKINS COIE LLP;PATENT-SEA, P.O. BOX 1247, SEATTLE, WA, 98111-1247, US

(21) Appl. No.: 11/233,814

(22) Filed: Sep. 23, 2005

(51) Int. Cl. A61N 1/00

(52) U.S. Cl. 607/2; 600/14

(57) ABSTRACT

Methods and apparatus are provided for inducing, monitoring and controlling renal neuromodulation using a pulsed electric field to effectuate electroporation or electrofusion. In some embodiments, tissue impedance, conductance or conductivity may be monitored to determine the effects of pulsed electric field therapy, e.g., to determine an extent of electroporation and its degree of irreversibility. Pulsed electric field electroporation of tissue causes a decrease in tissue impedance and an increase in tissue conductivity. If induced electroporation is reversible, upon cessation of the pulsed electric field, tissue impedance and conductivity should approximate baseline levels; however, if electroporation is irreversible, impedance and conductivity changes should persist. Thus, monitoring of impedance or conductivity may be utilized to determine the onset of electroporation and to determine the type or extent of electroporation. Furthermore, monitoring data may be used in one or more manual or automatic feedback loops to control the electroporation.

### CROSS REFERENCE TO RELATED APPLICATION

The present application claims priority to U.S. Provisional Application entitled METHODS AND APPARATUS FOR INDUCING, MONITORING AND CONTROLLING RENAL NUEROMODULATION, filed Sep. 20, 2005, (attorney reference no. 57856.8008.US00), the entirety of which is incorporated herein by reference.

### INCORPORATION BY REFERENCE

All publications and patent applications mentioned in this specification are herein incorporated by reference to the same extent as if each individual publication or patent application was specifically and individually indicated to be incorporated by reference.

### TECHNICAL FIELD

The present invention relates to methods and apparatus for renal neuromodulation. More particularly, the present invention relates to methods and apparatus for achieving renal neuromodulation via electroporation or electrofusion. Methods and apparatus for monitoring and controlling neuromodulation, as well as electrical waveforms for inducing such neuromodulation, are provided.

### BACKGROUND

Congestive Heart Failure (“CHF”) is a condition that occurs when the heart becomes damaged and reduces blood flow to the organs of the body. If blood flow decreases sufficiently, kidney function becomes impaired, which results in fluid retention, abnormal hormone secretions and increased constriction of blood vessels. These results increase the workload of the heart and further decrease the capacity of the heart to pump blood through the kidney and circulatory system.

It is believed that progressively decreasing perfusion of the kidney is a principal non-cardiac cause perpetuating the downward spiral of CHF. Moreover, the fluid overload and associated clinical symptoms resulting from these physiologic changes result in additional hospital admissions, poor quality of life and additional costs to the health care system.

In addition to their role in the progression of CHF, the kidneys play a significant role in the progression of Chronic Renal Failure (“CRF”), End-Stage Renal Disease (“ESRD”), hypertension (pathologically high blood pressure) and other cardio-renal diseases. The functions of the kidney can be summarized under three broad categories: filtering blood and excreting waste products generated by the body's metabolism; regulating salt, water, electrolyte and acid-base balance; and secreting hormones to maintain vital organ blood flow. Without properly functioning kidneys, a patient will suffer water retention, reduced urine flow and an accumulation of waste toxins in the blood and body. These conditions result from reduced renal function or renal failure (kidney failure) and are believed to increase the workload of the heart. In a CHF patient, renal failure will cause the heart to further deteriorate as fluids are retained and blood toxins accumulate due to the poorly functioning kidneys.

It has been established in animal models that the heart failure condition results in abnormally high sympathetic activation of the kidney. An increase in renal sympathetic nerve activity leads to vasoconstriction of blood vessels supplying the kidney, decreased renal blood flow, decreased removal of water and sodium from the body, and increased renin secretion. Reduction of sympathetic renal nerve activity, e.g., via denervation, may reverse these processes.

Applicants have previously described methods and apparatus for treating renal disorders by applying a pulsed electric field to neural fibers that contribute to renal function. See, for example, co-pending United States patent applications Ser. No. 11/129,765, filed on May 13, 2005, and Ser. No. 11/189,563, filed on Jul. 25, 2005, both of which are incorporated herein by reference in their entireties. A pulsed electric field (PEF) may initiate renal neuromodulation, e.g., denervation, via irreversible electroporation. The PEF may be delivered from apparatus positioned intravascularly, extravascularly, transvascularly or a combination thereof.

As used herein, electroporation and electropermeabilization are methods of manipulating the cell membrane or intracellular apparatus. For example, short, high-energy pulses open pores in cell membranes. The extent of porosity in the cell membrane (e.g., size and number of pores) and the duration of the pores (e.g., temporary or permanent) are a function of multiple variables, such as field strength, pulse width, duty cycle, field orientation, cell type and other parameters.

Cell membrane pores will generally close spontaneously upon termination of relatively lower strength fields or relatively shorter pulse widths (herein defined as “reversible electroporation”). However, each cell or cell type has a critical threshold above which pores do not close such that pore formation is no longer reversible; this result is defined as “irreversible electroporation,” “irreversible breakdown” or “irreversible damage.” At this point, the cell membrane ruptures and/or irreversible chemical imbalances caused by the high porosity occur. Such high porosity can be the result of a single large hole and/or a plurality of smaller holes.

When a PEF sufficient to initiate irreversible electroporation is applied to renal nerves and/or other neural fibers that contribute to renal neural functions, applicants believe that denervation induced by the PEF would result in increased urine output, decreased renin levels, increased urinary sodium excretion and/or controlled blood pressure that would prevent or treat CHF, hypertension, renal system diseases, and other renal anomalies. PEF systems could be used to modulate efferent or afferent nerve signals, as well as combinations of efferent and afferent signals.

A potential challenge of using PEF systems for treating renal disorders is monitoring the onset and the extent of electroporation, such as determining whether the electroporation is reversible or irreversible. Furthermore, it may also be challenging to selectively electroporate target cells without affecting other cells. For example, it may be desirable to irreversibly electroporate renal nerve cells that travel along or in proximity to renal vasculature, but it may not desirable to damage the smooth muscle cells of which the vasculature is composed. As a result, an overly aggressive course of PEF therapy may damage the renal vasculature, but an overly conservative course of PEF therapy may not achieve the desired renal neuromodulation.

In view of the foregoing, it would be desirable to provide methods and apparatus for monitoring and controlling renal neuromodulation, as well as electrical waveforms for achieving desired neuromodulatory effects.

### SUMMARY

The present invention provides methods and apparatus for monitoring and controlling pulsed electric field (PEF) renal neuromodulation, e.g., denervation, as well as PEF waveforms for inducing desired neuromodulatory effects. Embodiments of the invention may be configured for intravascular, extravascular and/or transvascular inducement, monitoring and control of renal neuromodulation.

Pulsed electric field parameters can include, but are not limited to, voltage, field strength, pulse width, pulse duration, the shape of the pulse, the number of pulses and/or the interval between pulses (e.g., duty cycle). Suitable field strengths include, for example, strengths of up to about 10,000 V/cm. Suitable pulse widths include, for example, widths of up to about 1 second. Suitable shapes of the pulse waveform include, for example, AC waveforms, sinusoidal waves, cosine waves, combinations of sine and cosine waves, DC waveforms, DC-shifted AC waveforms, RF waveforms, square waves, trapezoidal waves, exponentially-decaying waves, combinations thereof, etc. Suitable numbers of pulses include, for example, at least one pulse. Suitable pulse intervals include, for example, intervals less than about 10 seconds. These parameters are provided for the sake of illustration and should in no way be considered limiting. Any combination of parameters may be utilized, as desired. PEF waveforms for inducing desired neuromodulatory effects are provided.

Tissue impedance or conductivity may be monitored to determine the effects of pulsed electric field therapy, e.g., to determine an extent of electroporation and its degree of irreversibility. Pulsed electric field electroporation of tissue causes a decrease in tissue impedance and an increase in tissue conductivity. If induced electroporation is reversible, tissue impedance and conductivity should approximate baseline levels upon cessation of the pulsed electric field. However, if electroporation is irreversible, impedance and conductivity changes should persist after terminating the pulsed electric field. Thus, monitoring the impedance or conductivity of the target structure may be utilized to determine the onset of electroporation and to determine the type or extent of electroporation. Furthermore, monitoring data may be used in one or more manual or automatic feedback loops to control the electroporation.

Monitoring elements preferably are in electrical contact or in close proximity with the tissue being monitored. Thus, intravascular and/or extravascular monitoring elements may be utilized to monitor electroporation of smooth muscle cells and/or of the vessel wall. Likewise, transvascular and/or extravascular elements may be utilized to monitor electroporation of neural fibers that contribute to renal function and/or of surrounding tissues.

### BRIEF DESCRIPTION OF THE DRAWINGS

Several embodiments of the present invention will be apparent upon consideration of the following detailed description, taken in conjunction with the accompanying drawings, in which like reference characters refer to like parts throughout, and in which:

FIG. 1 is a schematic view illustrating human renal anatomy.

FIG. 2 is a schematic detail view showing the location of the renal nerves relative to the renal artery.

FIGS. 3A and 3B are schematic side- and end-views, respectively, illustrating orienting of electrical current flow for selectively affecting renal nerves.

FIG. 4 is a schematic view illustrating an exemplary extravascular method and apparatus for renal neuromodulation.

FIG. 5 is a schematic view illustrating an exemplary intravascular method and apparatus for renal neuromodulation.

FIG. 6 is a schematic flowchart illustrating methods of controlling pulsed electric field renal neuromodulation in response to electroporation monitoring feedback.

FIG. 7 is a side view, partially in section, of an alternative embodiment of the intravascular apparatus of FIG. 5 having independent monitoring elements, illustrating a method of monitoring and controlling PEF therapy at a target site within a patient's blood vessel.

FIGS. 8A and 8B are schematic side views of embodiments of a catheter with a centering element having both monitoring electrodes and PEF-delivery electrodes.

FIG. 9 is a schematic view of an exemplary circuit diagram for a PEF system comprising PEF-delivery electrodes and monitoring electrodes.

FIG. 10 is a side view, partially in section, of a catheter comprising combination monitoring and PEF-delivery electrodes.

FIG. 11 is a side view, partially in section, illustrating a method of using the apparatus of FIG. 10 to reduce vessel trauma in the event of a vessel spasm.

FIGS. 12A and 12B are side views, partially in section, illustrating a method of using the apparatus of FIG. 10 to ensure that the electrodes are not in contact with the vessel wall prior to, or during, PEF therapy.

FIG. 13 is a side view, partially in section, of a PEF system illustrating a method for transvascular monitoring and control of PEF therapy.

FIG. 14 is a side view, partially in section, of an alternative embodiment of the extravascular apparatus of FIG. 4 having independent monitoring elements, illustrating a method of extravascularly monitoring and controlling PEF therapy.

FIG. 15 is a side view, partially in section, of apparatus and a method for intravascular, extravascular and/or transvascular delivery, monitoring and control of PEF therapy.

FIG. 16 is a side view, partially in section, of a patient's renal vasculature, illustrating geometric variation along the vasculature.

FIG. 17 is a schematic graph illustrating an upward-sloping relationship between required applied voltage and vessel diameter for a desired field strength in target neural fibers that contribute to renal function.

FIG. 18 is a schematic view of an illustrative PEF waveform comprising a pulse train with one or more pulses of constant amplitude (voltage) or field strength, duration, and interval.

FIG. 19 is a schematic view of another illustrative PEF waveform comprising a pulse train with pulses of increasing field strength or amplitude.

FIG. 20 is a schematic view of yet another illustrative PEF waveform comprising a pulse train with pulses of increasing duration.

FIG. 21 is a schematic view of an illustrative PEF waveform comprising a pulse train with pulses of decreasing interval.

FIG. 22 is a schematic view of an illustrative PEF waveform comprising a pulse train of varying amplitude or field strength, duration, and/or interval.

FIG. 23 is a schematic view of an illustrative PEF waveform comprising a pulse train of increasing field strength and varying pulse duration and interval.

FIG. 24 is a schematic view of an illustrative PEF waveform comprising an AC pulse train of increasing amplitude.

FIGS. 25A and 25B are schematic views of individual AC pulses of illustrative PEF waveforms.

FIG. 26 is a schematic view of an illustrative PEF waveform comprising a composite AC and DC pulse train.

FIG. 27 is a schematic view of an alternative composite AC and DC pulse train.

### DETAILED DESCRIPTION

A. Overview

The present invention relates to methods and apparatus for monitoring and controlling pulsed electric field (PEF) renal neuromodulation, e.g., denervation, as well as PEF waveforms for inducing desired neuromodulatory effects. Embodiments of the invention may be configured for intravascular, extravascular and/or transvascular inducement, monitoring and control of renal neuromodulation. A combination of intravascular, extravascular and/or transvascular elements optionally may be utilized. The apparatus and methods described herein may exploit any suitable electrical signal or field parameters, e.g., any electric field that will achieve the desired neuromodulation (e.g., electroporative effect). To better understand the structures of devices of the present invention and the methods of using such devices for neuromodulation and monitoring, it is instructive to examine the renal anatomy in humans.

B. Selected Embodiments of Methods for Neuromodulation

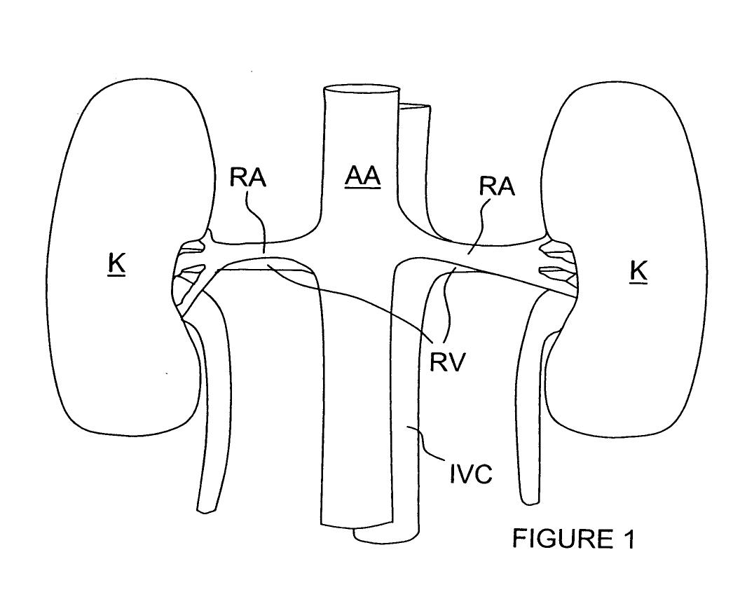

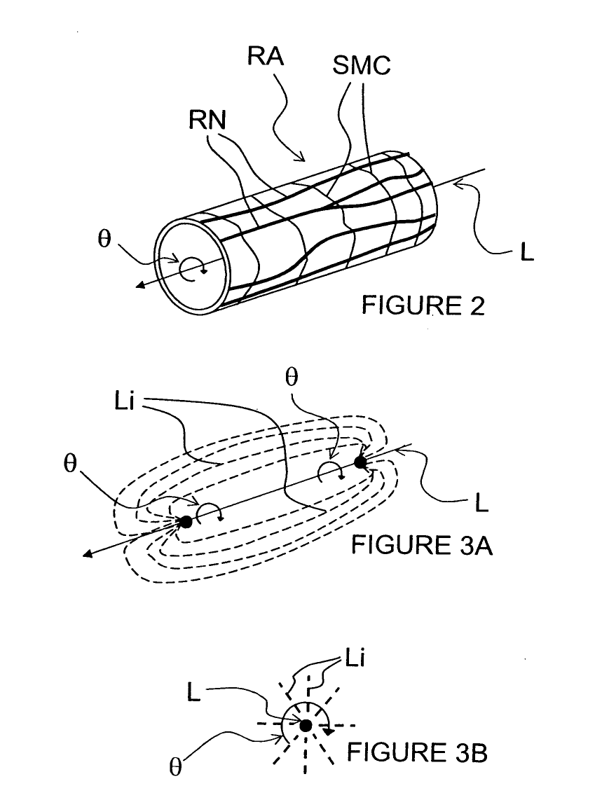

With reference now to FIG. 1, the human renal anatomy includes kidneys K that are supplied with oxygenated blood by renal arteries RA, which are connected to the heart by the abdominal aorta AA. Deoxygenated blood flows from the kidneys to the heart via renal veins RV and the inferior vena cava IVC. FIG. 2 illustrates a portion of the renal anatomy in greater detail. More specifically, the renal anatomy also includes renal nerves RN extending longitudinally along the lengthwise dimension L of renal artery RA generally within the adventitia of the artery. The renal artery RA has smooth muscle cells SMC that surround the arterial circumference and spiral around the angular axis θ of the artery. The smooth muscle cells of the renal artery accordingly have a lengthwise or longer dimension extending transverse (i.e., non-parallel) to the lengthwise dimension of the renal artery. The misalignment of the lengthwise dimensions of the renal nerves and the smooth muscle cells is defined as “cellular misalignment.”

Referring to FIG. 3, the cellular misalignment of the renal nerves and the smooth muscle cells may be exploited to selectively affect renal nerve cells with reduced effect on smooth muscle cells. More specifically, because larger cells require less energy to exceed the irreversibility threshold of electroporation, several embodiments of electrodes of the present invention are configured to align at least a portion of an electric field generated by the electrodes with or near the longer dimensions of the cells to be affected. In specific embodiments, the device has electrodes configured to create an electrical field aligned with or near the lengthwise dimension L of the renal artery RA to affect renal nerves RN. By aligning an electric field so that the field preferentially aligns with the lengthwise aspect of the cell rather than the diametric or radial aspect of the cell, lower field strengths may be used to affect target neural cells, e.g., to necrose or fuse the target cells and/or to induce apoptosis. As mentioned above, this is expected to reduce power consumption and mitigate effects on non-target cells in the electric field.

Similarly, the lengthwise or longer dimensions of tissues overlying or underlying the target nerve are orthogonal or otherwise off-axis (e.g., transverse) with respect to the longer dimensions of the nerve cells. Thus, in addition to aligning the PEF with the lengthwise or longer dimensions of the target cells, the PEF may propagate along the lateral or shorter dimensions of the non-target cells (i.e., such that the PEF propagates at least partially out of alignment with non-target smooth muscle cells SMC). Therefore, as seen in FIG. 3, applying a PEF with propagation lines Li generally aligned with the longitudinal dimension L of the renal artery RA is expected to preferentially cause electroporation, electrofusion, denervation or other neuromodulation in cells of the target renal nerves RN without unduly affecting the non-target arterial smooth muscle cells SMC. The pulsed electric field may propagate in a single plane along the longitudinal axis of the renal artery, or may propagate in the longitudinal direction along any angular segment θ through a range of 0°-360°.

A PEF system placed exterior to, within, and/or at least partially across the wall of the renal artery may propagate an electric field having a longitudinal portion that is aligned to run with the longitudinal dimension of the artery in the region of the renal nerves RN and the smooth muscle cell SMC of the vessel wall so that the wall of the artery remains at least substantially intact while the outer nerve cells are destroyed or fused. Monitoring elements may be utilized to assess an extent of, e.g., electroporation, induced in renal nerves and/or in smooth muscle cells, as well as to adjust PEF parameters to achieve a desired effect.

C. Exemplary Embodiments of Systems and Additional Methods for Neuromodulation

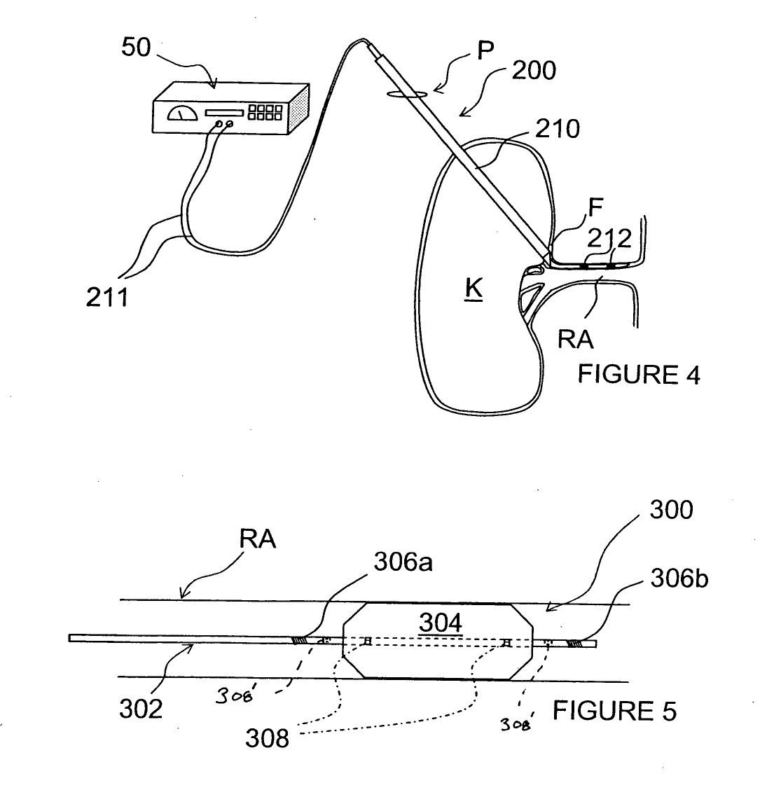

With reference to FIGS. 4 and 5, exemplary embodiments of PEF systems and methods are described. FIG. 4 shows one embodiment of an extravascular pulsed electric field apparatus 200 that includes one or more electrodes configured to deliver a pulsed electric field to renal neural fibers to achieve renal neuromodulation. The apparatus of FIG. 4 is configured for temporary extravascular placement; however, it should be understood that partially or completely implantable extravascular apparatus additionally or alternatively may be utilized.

In FIG. 4, apparatus 200 comprises a laparoscopic or percutaneous PEF system having probe 210 configured for insertion in proximity to the track of the renal neural supply along the renal artery or vein or hilum and/or within Gerota's fascia under, e.g., CT or radiographic guidance. At least one electrode 212 is configured for delivery through probe 210 to a treatment site for delivery of pulsed electric field therapy. The electrode(s) 212 may comprise a catheter and are electrically coupled to pulse generator 50 via wires 211. In an alternative embodiment, the distal section of probe 210 may comprise the at least one electrode 212, and the probe may have an electrical connector to couple the probe to the pulse generator 50 for delivering a PEF to the electrode(s) 212.

The pulsed electric field generator 50 is located external to the patient. The generator, as well as any of the PEF-delivery electrode embodiments described herein, may be utilized with any embodiment of the present invention for delivery of a PEF with desired field parameters. It should be understood that PEF-delivery electrodes of embodiments described hereinafter may be electronically connected to the generator even though the generator is not explicitly shown or described with each embodiment.

The electrode(s) 212 can be individual electrodes that are electrically independent of each other, a segmented electrode with commonly connected contacts, or a continuous electrode. A segmented electrode may, for example, be formed by providing a slotted tube fitted onto the electrode, or by electrically connecting a series of individual electrodes. Individual electrodes or groups of electrodes 212 may be configured to provide a bipolar signal. The electrodes 212 may be dynamically assignable to facilitate monopolar and/or bipolar energy delivery between any of the electrodes and/or between any of the electrodes and an external ground pad. Such a ground pad may, for example, be attached externally to the patient's skin, e.g., to the patient's leg or flank. In FIG. 4, the electrodes 212 comprise a bipolar electrode pair. The probe 210 and the electrodes 212 may be similar to the standard needle or trocar-type used clinically for pulsed RF nerve block, such as those sold by Valleylab (a division of Tyco Healthcare Group LP) of Boulder, Colo. Alternatively, the apparatus 200 may comprise a flexible and/or custom-designed probe for the renal application described herein.

In FIG. 4, the percutaneous probe 210 has been advanced through percutaneous access site P into proximity with renal artery RA. The probe pierces Gerota's fascia F, and the electrodes 212 are advanced into position through the probe and along the annular space between the patient's artery and fascia. Once properly positioned, pulsed electric field therapy may be applied to target neural fibers across the bipolar electrodes 212. Such PEF therapy may, for example, denervate the target neural fibers through irreversible electroporation. Electrodes 212 optionally also may be used to monitor the electroporative effects of the PEF therapy, as described hereinbelow. After treatment, the apparatus 200 may be removed from the patient to conclude the procedure.

Referring now to FIG. 5, another embodiment of an intravascular PEF system is described. This embodiment includes an apparatus 300 comprising a catheter 302 having a centering element 304 (e.g., a balloon, an expandable wire basket, other mechanical expanders, etc.), shaft electrodes 306a and 306b disposed along the shaft of the catheter, and optional radiopaque markers 308 disposed along the shaft of the catheter in the region of the centering element 304. The electrodes 306a-b, for example, can be arranged such that the electrode 306a is near a proximal end of the centering element 304 and the electrode 306b is near the distal end of the centering element 304. Electrodes 306 are electrically coupled to pulse generator 50 (see FIG. 4), which is disposed external to the patient, for delivery of PEF therapy. The radiopaque markers can alternatively be located along the shaft outside of the centering element 304 as shown by optional markers 308′, or the electrodes can be made from a radiopaque material (e.g., platinum) to eliminate the separate markers 308.

Electrodes 306 can be individual electrodes (i.e., independent contacts), a segmented electrode with commonly connected contacts, or a single continuous electrode. Furthermore, electrodes 306 may be configured to provide a bipolar signal, or electrodes 306 may be used together or individually in conjunction with a separate patient ground for monopolar use. When centering element 304 comprises an inflatable balloon, the balloon may serve as both a centering element for electrodes 306 and as an electrical insulator for directing an electric field delivered across the electrodes, e.g., for directing the electric field into or across the vessel wall for modulation of target neural fibers. Electrical insulation provided by element 304 may reduce the magnitude of applied voltage or other parameters of the pulsed electric field necessary to achieve desired field strength at the target tissue.

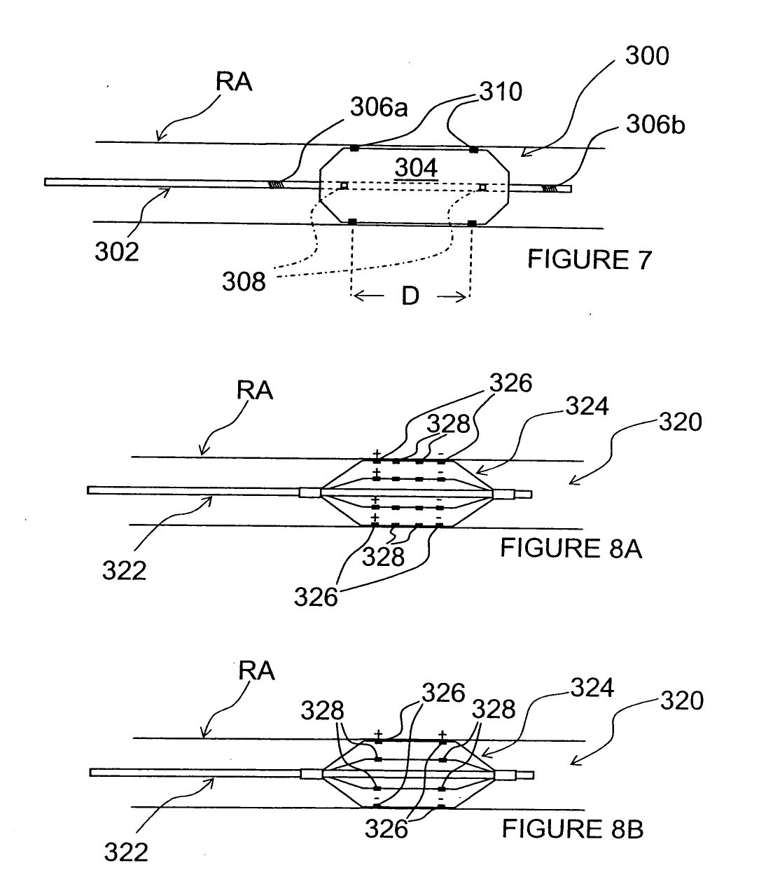

As an alternative or in addition to placement of electrodes 306 along the central shaft of catheter 302, electrodes 306 may be attached to centering element 304 such that they contact the wall of renal artery RA (e.g., surface contact and/or penetration). In such a variation, the electrodes may, for example, be affixed to the inside surface, outside surface or at least partially embedded within the wall of the centering element. The electrodes optionally may be used to monitor the effects of PEF therapy, as described hereinafter. As it may be desirable to reduce or minimize physical contact between the PEF-delivery electrodes and the vessel wall during delivery of PEF therapy in order to reduce the potential for injuring the wall. The electrodes 306 may, for example, be a first set of electrodes attached to the shaft of the catheter for delivering the PEF therapy, and the device may further include a second set of electrodes optionally attached to centering element 304 for monitoring the effects of PEF therapy delivered via electrodes 306, as discussed hereinbelow with respect to FIG. 7.

In use, catheter 302 may be delivered to renal artery RA as shown, or it may be delivered to a renal vein or to any other vessel in proximity to neural tissue contributing to renal function, in a low profile delivery configuration, for example, through a guide catheter. Once positioned within the renal vasculature, optional centering element 304 may be expanded into contact with an interior wall of the vessel. A pulsed electric field then may be generated by the PEF generator 50, transferred through catheter 302 to electrodes 306, and delivered via the electrodes 306 across the wall of the artery. The PEF therapy modulates the activity along neural fibers that contribute to renal function, e.g., denervates the neural fibers. This may be achieved, for example, via irreversible electroporation, electrofusion and/or inducement of apoptosis in the nerve cells. In many applications, the electrodes are arranged so that the pulsed electric field is aligned with the longitudinal dimension of the renal artery to facilitate modulation of renal nerves with little effect on non-target smooth muscle cells or other cells.

It is expected that PEF therapy, whether delivered extravascularly, intravascularly, transvascularly or a combination thereof, will alleviate clinical symptoms of CHF, hypertension, renal disease and/or other cardio-renal diseases for a period of months, potentially up to six months or more. This time period might be sufficient to allow the body to heal; for example, this period might reduce the risk of CHF onset after an acute myocardial infarction, thereby alleviating a need for subsequent re-treatment. Alternatively, as symptoms reoccur, or at regularly scheduled intervals, the patient might return to the physician for a repeat therapy.

The apparatus described above with respect to FIGS. 4 and 5 may be used to quantify the efficacy, extent, or cell selectivity of PEF therapy to monitor and/or control the therapy. When a pulsed electric field initiates electroporation, the impedance of the electroporated tissue begins to decrease and the conductivity of the tissue begins to increase. If the electroporation is reversible, the tissue electrical parameters will return or approximate baseline values upon cessation of the PEF. However, if the electroporation is irreversible, the changes in tissue parameters will persist after termination of the PEF. These phenomena may be utilized to monitor both the onset and the effects of PEF therapy. For example, electroporation may be monitored directly using, for example, conductivity measurements or impedance measurements, such as Electrical Impedance Tomography (“EIT”) and/or other electrical impedance/conductivity measurements like an electrical impedance or conductivity index. Such electroporation monitoring data may be used in one or more feedback loops to better control delivery of PEF therapy.

For the purposes of the present invention, the imaginary part of impedance is ignored and impedance is defined as voltage divided by current, while conductance is defined as the inverse of impedance (i.e., current divided by voltage), and conductivity is defined as conductance per unit distance. The distance between monitoring electrodes preferably is known prior to therapy delivery and may be used to determine conductivity from impedance or conductance measurements.

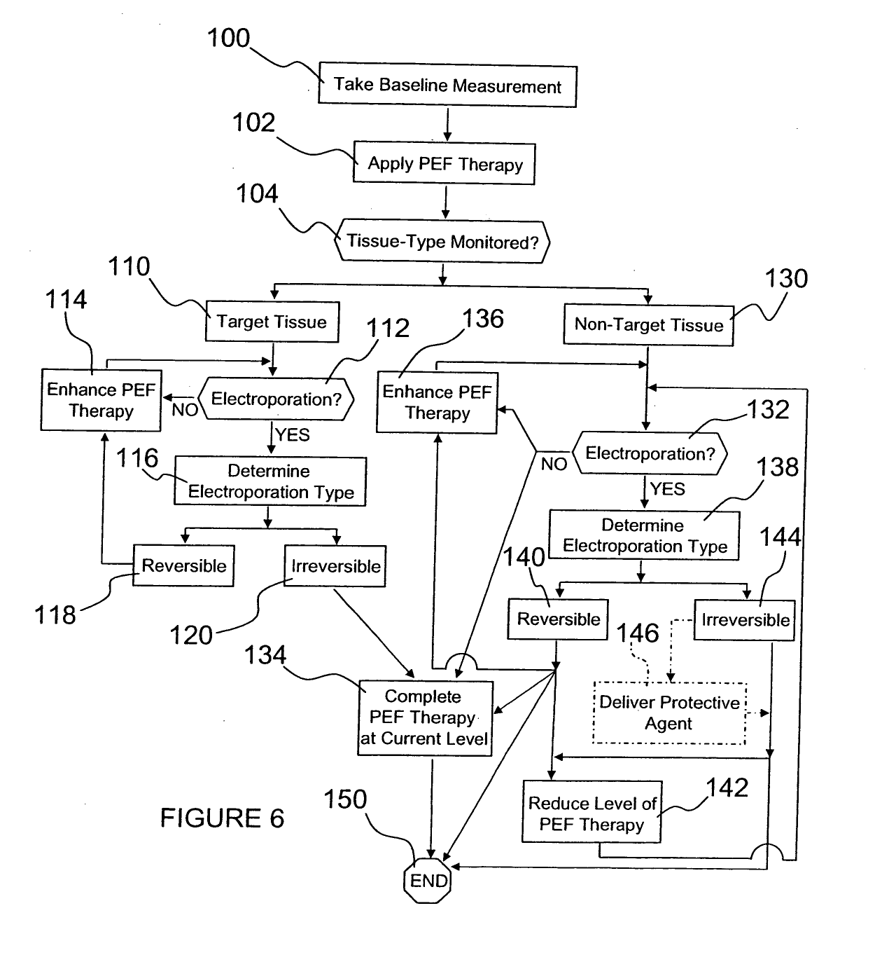

FIG. 6 provides a schematic flowchart illustrating methods of controlling pulsed electric field renal neuromodulation in response to electroporation monitoring feedback. These methods may be utilized intravascularly, extravascularly, transvascularly or a combination thereof. In FIG. 6, Step 100 comprises taking a baseline measurement of impedance and/or conductivity for the tissue being monitored, e.g., by emitting a low voltage pulse through the tissue (for example, a voltage less than about 20 volts) and measuring the response. This baseline may be utilized as a reference against which changes in impedance or conductivity may be compared upon application of a pulsed electric field to the tissue being monitored. As discussed previously, electroporation of tissue causes tissue impedance to decrease and causes tissue conductivity to increase.

With the baseline established, Step 102 comprises applying PEF therapy in the vicinity of the tissue being monitored. As seen in Step 104, the desired response of monitored tissue to such therapy depends upon whether the tissue being monitored is the target tissue of Routine 110 or the non-target tissue of Routine 130. Generally, it is desirable to electroporate or irreversibly electroporate the target tissue of Routine 110, while it may be undesirable to electroporate or irreversibly electroporate the non-target tissue of Routine 130. The target tissue of Routine 110 may comprise, for example, neural fibers that contribute to renal function, while the non-target tissue of Routine 130 may comprise, for example, the interior or exterior wall of renal vasculature and/or of smooth muscle cells.

Monitoring elements preferably are in physical contact or in close proximity with the tissue being monitored. For example, non-target tissue may be monitored intravascularly or extravascularly; i.e., within, or exterior and in close proximity to, renal vasculature. Target tissue may, for example, be monitored extravascularly or may be monitored transvascularly, for example, by placing monitoring elements in the vascular adventitia. Other alternative monitoring arrangements may be provided.

For the target tissue of Routine 110, after application of PEF therapy during Step 102, Step 112 comprises monitoring the impedance and/or conductivity of the target tissue, e.g., emitting a low voltage pulse through the tissue and measuring the response, to determine whether the tissue has been electroporated. As mentioned, electroporation increases tissue conductivity and decreases tissue impedance. If the tissue has not been electroporated, then PEF therapy should be enhanced, as in Step 114. PEF enhancement comprises increasing the strength, intensity, duration, positioning, etc., of any of the parameters of the pulsed electric field that contribute to inducement of tissue electroporation. Additionally, PEF can be enhanced by providing agents that impart beneficial properties to the tissue (e.g., conductivity). Suitable agents include saline, hypertonic saline, and other compounds.

If the target tissue has been electroporated, Step 116 comprises determining what type of electroporation has occurred, i.e. reversible electroporation of Step 118 or irreversible electroporation of Step 120. For example, an absolute or a threshold relative change in tissue impedance or conductivity from the baseline measurement taken in Step 100 may be indicative of the type of electroporation. Additionally or alternatively, the persistence of changes in monitored electrical parameters after cessation of PEF therapy may be used to determine electroporation type. For example, changes in impedance or conductivity that persist after termination of the PEF are indicative of the irreversible electroporation of Step 120; conversely, a return of impedance or conductivity to or approximate the baseline value obtained during Step 100 is indicative of the reversible electroporation of Step 118.

For target tissue, if it is determined that induced electroporation is reversible, then PEF therapy should be enhanced, as in the feedback loop of Step 114, until irreversible electroporation is achieved. Likewise, if it is determined that the electroporation is irreversible, then the procedure may be completed at its current level, as in Step 134, then concluded in Step 150.

If the tissue being monitored is the non-target tissue of Routine 130, Step 132 comprises determining whether the PEF therapy of Step 102 has induced or is presently inducing electroporation in the non-target tissue. This may be achieved by monitoring the impedance or conductivity of the non-target tissue of Routine 130, e.g., by emitting a low voltage pulse through the tissue and measuring the response, and comparing measured values to the baseline measurement of Step 100. Measurements preferably are taken and analyzed in real time.

As discussed, electroporation, and especially irreversible electroporation, generally is not desirable in non-target tissue. However, reversible electroporation and/or a limited amount of irreversible electroporation of non-target tissue may be acceptable in order to irreversibly electroporate the target tissue. The potential for undesirably injuring the non-target tissue should be weighed against the expected benefits of irreversibly electroporating the target tissue.

If it is determined that electroporation of the non-target tissue has not occurred, then the medical practitioner (or, alternatively, the system in an automatic feedback loop via pre-programmed instructions) has a few options. The practitioner may complete PEF therapy at the current electrical parameters without altering the position of the PEF system and apparatus, as in Step 134. This may be desirable, for example, if the PEF therapy is of sufficient magnitude and is delivered in a manner sufficient to initiate irreversible electroporation in target tissue without electroporating the non-target tissue. After completion of the PEF therapy, the procedure may be concluded, as in Step 150.

The practitioner alternatively may enhance the PEF therapy, as in the feedback loop of Step 136. This may be desirable, for example, if the PEF therapy is insufficient to initiate irreversible electroporation in the target tissue, as determined, for example, via (a) optional concurrent monitoring of target tissue, (b) predictions from modeling simulations, (c) statistical reference to previously conducted PEF therapy with similar waveform parameters, etc. After enhancement of the PEF therapy, Step 132 may be repeated to determine whether the enhanced PEF therapy induces electroporation in the non-target tissue of Routine 130.

If, Step 132 establishes that PEF therapy has induced electroporation in the non-target tissue (either at the initial PEF therapy levels of Step 102 or after enhancement of the PEF therapy via Step 136), then Step 138 comprises determining the type of electroporation that has occurred. Step 138, for example, can utilize the techniques described previously with respect to target tissue monitoring. If it is determined that the electroporation comprises the reversible electroporation of Step 140, then the medical practitioner or an automated control system has four options. The first option is to immediately terminate PEF therapy, as in Step 150. This is the most conservative course of action for reducing potential injury to the non-target tissue monitored in Step 130. However, this may not be sufficient to achieve a desired level of, e.g., irreversible electroporation in the target tissue of Step 110.

Another option is to proceed to the feedback loop of Step 142, which comprises reducing the level or magnitude of the PEF therapy along non-target tissue. This may comprise repositioning elements of the PEF system and/or altering electrical parameters of the pulsed electric field. Reducing the magnitude of the PEF therapy may be sufficient to reduce or stop electroporation of the non-target tissue. However, reductions in the magnitude of the therapy should be weighed against the effect of the reductions on desired electroporation of target tissue. Overly aggressive reduction in the pulsed electric field may negate the field's ability to advantageously electroporate the target tissue of Step 110.

Alternatively, PEF therapy may be completed at the then-current levels or magnitude, as in Step 134. If the monitored electrical parameters indicate that electroporation of the non-target tissue is reversible, then the potential for sustained injury to the non-target tissue of Step 130 associated with continuing the PEF therapy may be relatively low and may support continued therapy at the then-current levels, as needed, to achieve desired effects on the target tissue of Step 110. However, continuation of the PEF therapy should be weighed against the potential for non-target tissue injury. After completion of PEF therapy under Step 134, the procedure may be concluded in Step 150.

Another alternative is to enhance the magnitude of PEF therapy, as in the feedback loop of Step 136, then repeat the electroporation monitoring and decision process to ensure that the new level of PEF therapy still has an acceptably low potential for inducing sustained injury in the non-target tissue. It may, for example, be desirable to enhance PEF therapy until a therapy level sufficient to induce irreversible electroporation in the target tissue of Step 110 is achieved. Alternatively or additionally, PEF therapy may be enhanced until the non-target tissue of Step 130 is reversibly electroporated and/or until the monitored parameter(s) of the non-target tissue are altered by a threshold amount, e.g., until a threshold change in tissue impedance is observed.

The PEF therapy optionally may be progressively and gradually ramped up from a low level to the desired level while monitoring non-target tissue in order to reduce the potential for sustained injury to the non-target tissue. Ramping of the PEF therapy may be discontinued or reversed at any time, for example, when the potential for sustained injury to the non-target tissue outweighs the potential benefits of therapy at a given level of PEF magnitude. Additional PEF waveforms, as well as techniques for altering the waveforms in response to monitoring data, are described hereinafter.

If it is determined that electroporation of the non-target tissue of Step 130 comprises undesirable irreversible electroporation, as in Step 144, then the medical practitioner or automated control system (e.g., auto-feedback loop) may reduce the level of PEF therapy, preferably to a level that does not continue to irreversibly electroporate the non-target tissue, as in the feedback loop of Step 142. Alternatively, the medical practitioner or automated control system may halt PEF therapy and conclude the procedure, as in Step 150. In some embodiments, such reduction or termination of PEF therapy may be implemented automatically by the PEF system whenever irreversible electroporation of non-target tissue is observed. The medical practitioner optionally might deliver, e.g., inject, a protective agent, such as Poloxamer-188, to irreversibly electroporated non-target tissue in order to reduce the potential for, or the degree of, sustained injury to the non-target tissue, as in Step 146.

With reference now to FIG. 7, an alternative embodiment of previously-described apparatus 300 comprising monitoring elements is described. In FIG. 7, apparatus 300 comprises monitoring electrodes 310 coupled to centering element 304. Monitoring electrodes 310 may be utilized to monitor the effects of PEF therapy delivered via electrodes 306, e.g., by emitting a low voltage pulse across monitoring electrodes 310 and through the monitored tissue, and then measuring the impedance or conductivity of the monitored tissue. The separation distance D between the monitoring electrodes 310 preferably is known in order to facilitate determination of tissue conductivity (conductance per unit distance) from tissue conductance or impedance measurements. Electrodes 310 optionally may be electrically coupled to pulse generator 50, for example, in a feedback loop, and/or may be electrically coupled to other external element(s) for emitting the low voltage monitoring pulse, or for recording, displaying and/or processing monitoring data collected via the electrodes. Although the apparatus 300 shown in FIG. 7 comprises separate electrode pairs for PEF therapy delivery and for monitoring of PEF effects, the same electrodes alternatively may be used both for delivery of PEF therapy and for monitoring of the effects of such therapy.

In use, electrodes 310 directly contact the vessel wall, as seen in FIG. 7. A baseline conductivity or impedance measurement may be made to determine steady-state tissue parameters prior to PEF therapy, e.g., by emitting a low voltage pulse across the monitoring electrodes and through the tissue, and then measuring the response of the monitored tissue. Once the baseline has been established, a pulse train may be applied to the tissue via bipolar electrode pair 306 to cause electroporation, and the effects of such electroporation may be monitored via monitoring electrodes 310, e.g., by applying another low voltage pulse across the electrodes 310 and examining changes in tissue impedance or conductivity from the baseline values.

The time between each PEF pulse, or after two or more PEF pulses, optionally may be sufficient to assess the status of the electroporative effect on the vessel wall via the monitoring electrodes. Monitoring alternatively or additionally may be conducted before and after application of PEF therapy to ensure the desired effect. To prevent circuit disruption, monitoring electrodes 310 optionally may be electrically disconnected during activation of PEF-delivery electrodes 306.

In some embodiments, it may be desirable to avoid irreversible electroporation of the vessel wall. In such an embodiment, PEF therapy may be interrupted should a target level of impedance decrease or conductivity increase occur at the vessel wall. This would provide a feedback system to ensure that non-target cells are not irreversibly electroporated during irreversible electroporation of target cells, such as nerve cells that contribute to renal function.

Additionally or alternatively, a treatment algorithm may be employed wherein the PEF pulse train starts out with a relatively small field strength [voltage/unit distance] that gradually increases based upon monitoring feedback. For example, the treatment may be initiated with relatively small field strength, E₁, delivered by electrodes 306. If monitoring data collected via monitoring electrodes 310 indicates that the application of E₁ does not alter the impedance or conductivity of the vessel wall, it is unlikely that electroporation has been initiated in the vessel wall. Thus, the field strength may be increased to E₂, etc., until electroporation is initiated. As electroporation is initiated, the impedance decreases and the conductivity increases, but these parameters should recover their baselines values once the field is no longer applied. If this recovery occurs, electroporation was reversible and an even larger field, E₃, optionally may be applied. Alternatively, a percent change or an absolute change in tissue impedance or conductivity may be used which predicts the outcome of reversible or irreversible electroporation. This monitoring technique may be used to prevent or reduce unwanted injury to the vessel wall. The flowchart of FIG. 6 may be used as a decision tree to guide delivery of the ramping pulsed electric field.

With reference now to FIGS. 8, an alternative apparatus 320 for delivering and monitoring PEF therapy is described. Apparatus 320 comprises intravascular catheter 322 having centering element 324 with PEF-delivery electrodes 326 and monitoring electrodes 328. Centering element 324 may, for example, comprise a balloon or an expandable basket. FIG. 8A illustrates an embodiment wherein the source and sink of the PEF-delivery electrodes 326 and the electrode pairs of the monitoring electrodes 328 are separated from one another along the longitudinal axis of centering element 324, and FIG. 8B illustrates an embodiment wherein the electrode pairs are separated from one another along the radial axis of the centering element. As will be apparent, the source and sink of the PEF-delivery electrodes, and/or the electrodes of the monitoring electrode pairs, may be separated from one another along both the longitudinal and the radial axis of the centering element. As also will be apparent, the electrodes alternatively may be utilized in an extravascular or transvascular embodiment of the present invention; for example, the electrodes may be integrated into an external cuff electrode.

In FIGS. 8A and 8B, apparatus 320 comprises a plurality of PEF-delivery electrodes and a plurality of monitoring electrodes. In such a configuration, a multiplexer may be used to deliver PEF therapy across a desired pair or plurality of PEF-delivery electrodes 326; likewise, a multiplexer may be utilized to deliver the low voltage signal and facilitate conductivity or impedance measurements across a desired pair or plurality of monitoring electrodes 328. A matrix of PEF therapy and/or monitoring configurations may be used to facilitate PEF delivery or monitoring as desired. Such a multiplexer may be used to deliver PEF therapy with other embodiments of apparatus set forth herein.

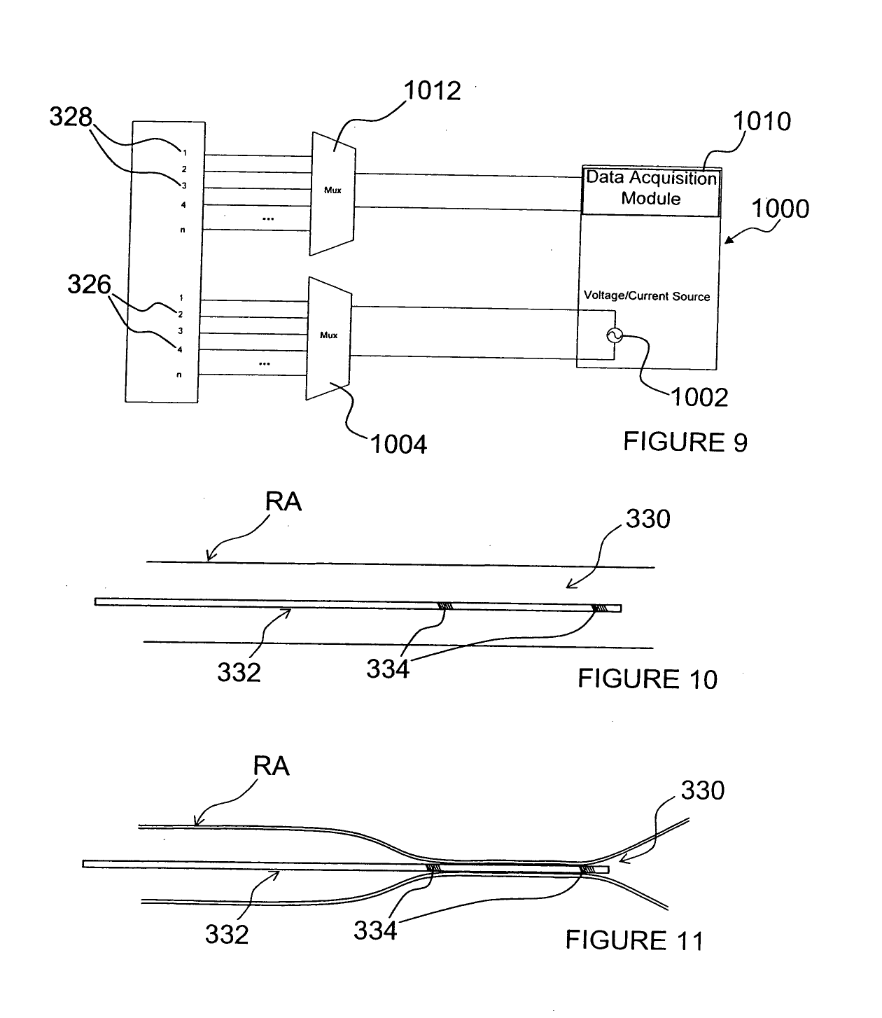

FIG. 9 illustrates an embodiment of a circuit diagram for such a multiplexed configuration. External control apparatus 1000, which may, for example, comprise an embodiment of pulse generator 50 described previously, a computer, a data acquisition module, etc., comprises voltage or current source 1002 coupled to multiplexer 1004. The multiplexer routes PEF waveforms generated by source 1002 to desired PEF-delivery electrodes 326. Apparatus 1000 further comprises data acquisition module 1010 coupled to multiplexer 1012, which delivers the low voltage signal, then measures and monitors data from selected monitoring electrodes 328.

Multiplexed PEF therapy delivery and monitoring facilitates optional formation of a 3-dimensional conductivity or impedance map based on multiple electrode measurements. This map may be used to determine the type and/or extent of electroporation throughout the target region, rather than providing an average conductivity or impedance value indicative of overall tissue characteristics. Multiplexed therapy and monitoring may, for example, comprise switching through each PEF-delivery and/or monitoring electrode pair. Data acquisition module 1010 may measure the potential across all pairs, or a desired subset, of the monitoring electrodes.

In embodiments that monitor PEF therapy with the same electrodes that deliver the PEF, conductivity or impedance may be determined by measuring the current draw across the electrodes under a voltage source, or by measuring the voltage applied under a constant current output. A differential potential measurement additionally or alternatively may be taken across the electrodes by delivering a low voltage signal before, during (e.g., between pulses) or after PEF delivery, as with the stand-alone monitoring electrodes.

Referring now to FIG. 10, another use for impedance or conductivity monitoring may be to ensure that intravascular electrodes used for applying a PEF pulse train do not come into direct contact with the vessel wall. In some indications, it may be desirable to position the PEF-delivery electrodes such that there is at least some spacing from the vessel wall, for example, to reduce a potential for injury to the vessel wall during PEF therapy. As seen in FIG. 10, apparatus 330 comprises catheter 332 having bipolar electrode pair 334 that may be used both for PEF therapy delivery and for monitoring of tissue parameters at a treatment site before, during or after PEF therapy.

Since the impedance of blood generally is lower than the impedance of the vessel wall, the observed impedance discontinuity between the blood and the wall may be used as a feedback mechanism to determine whether the electrodes are in contact with the vessel wall, i.e., to ensure proper positioning of the electrodes prior to or during delivery of PEF therapy. In FIG. 10, the catheter 332 is positioned such that electrodes 334 do not contact the wall of renal artery RA. Thus, impedance measurements across the electrodes are relatively low and indicate that the electrodes generally are not in contact with the vessel wall. If the electrodes were to contact the wall of the vessel before or during PEF therapy, the increased impedance levels would indicate such contact and optionally might immediately terminate or preclude PEF therapy until relatively lower impedance values are once again observed.

As seen in FIG. 11, in some patients, PEF therapy might induce spasm in the vessel wall. If this were to occur, the vessel might prolapse against catheter 332. The increased impedance observed across electrodes 334 would indicate that the electrodes were in contact with the vessel wall. In response, PEF therapy could be terminated, either manually or automatically. Termination of the pulsed electric field might reduce injury to the vessel wall, as compared to continued delivery of PEF therapy.

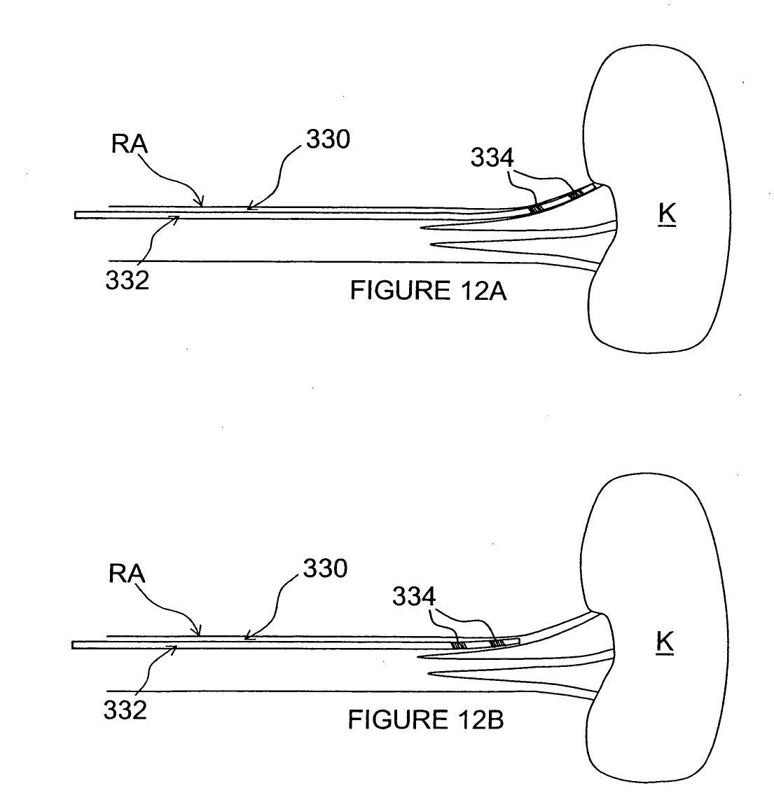

Referring now to FIGS. 12, impedance measurements also may be used to ensure that catheter 332 isn't positioned in a vessel too small to accommodate electrodes 334 without the electrodes contacting the vessel wall. As seen in FIG. 12A, catheter 332 is disposed in a branch of renal artery RA that is too small to accommodate the electrodes. The increased impedance levels associated with contacting the vessel wall and observed across electrodes 334 would indicate to a medical practitioner that the catheter was not properly positioned for PEF therapy. In some embodiments, apparatus 330 may comprise features that preclude delivery of a pulsed electric field when electrodes 334 are in contact with the vessel wall. As seen in FIG. 12B, the catheter may be withdrawn to a more proximal position within the artery where the electrodes do not contact the vessel wall; the relatively low impedance levels observed across the electrodes when positioned as in FIG. 12B would indicate that PEF therapy could proceed.

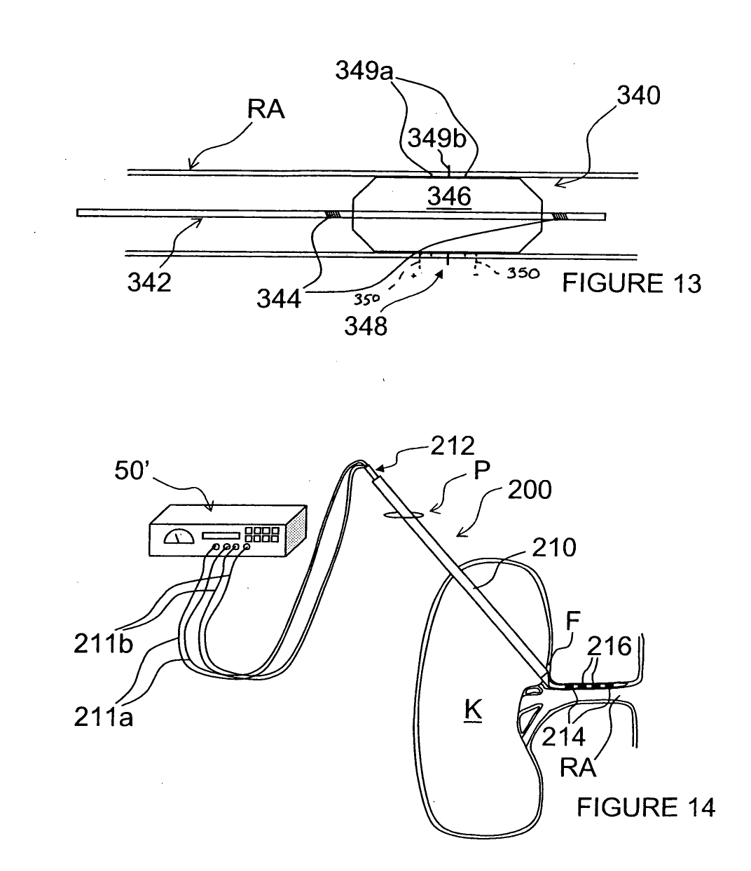

With reference now to FIG. 13, it may be desirable to monitor electrical parameters within or external to the vessel wall, for example, within the adventitia of the vessel wall. Neural fibers that contribute to renal function may be positioned in or around the adventitia. Apparatus 340 of FIG. 13 is configured for intravascular delivery to a treatment site and for transvascular monitoring of PEF therapy. Apparatus 340 comprises catheter 342 having PEF-delivery electrodes 344 coupled to the shaft of catheter 342, as well as micro-puncture needle electrodes 348 coupled to expandable centering element 346.

Needle electrodes 348 may be configured to penetrate to various depths within a vessel wall for monitoring the impedance or conductivity of target or non-target tissue within or exterior to the wall, for example, for monitoring smooth muscle tissue of the vessel wall, for monitoring renal nerves in the adventitia, or for monitoring surrounding tissue, e.g., surrounding fat. The micro-puncture needle electrodes illustratively comprise non-target tissue monitoring electrodes 349a that are configured to penetrate within the vessel wall for monitoring of tissue within the wall, such as smooth muscle cells, as well as target tissue monitoring electrodes 349b that are configured to penetrate deeper into the vascular adventitia for monitoring of the neural fibers or tissue continuing neural fibers that contribute to renal function. In addition, or as an alternative, to their use in monitoring electrical characteristics of tissue, micro-puncture needles 348 may be used to inject agents transvascularly, such as protective agents, neurotoxins, PEF enhancing agents (e.g., saline or hypertonic saline), etc. Additional and alternative agents are described hereinbelow.

In use, catheter 342 is delivered to a treatment site, for example, within a patient's renal artery. The centering element 346 is expanded into contact with the wall of the vessel, which acts to center PEF-delivery electrodes 344 within the vessel, as well as to transvascularly position micro-puncture needle electrodes 348. Baseline measurements of impedance or conductivity are obtained via needle electrodes 348, i.e., for the non-target tissue with electrodes 349a and for the target tissue with electrodes 349b. PEF therapy then is delivered via electrodes 344, and the therapy is monitored and controlled via feedback data received from electrodes 348, for example, according to the guidelines of the flowchart of FIG. 6. As mentioned, agents additionally or alternatively may be injected through electrodes 348. After completion of the PEF therapy, balloon 346 is deflated (the centering element is collapsed), which removes the needle electrodes from the vessel wall, and catheter 342 is removed from the patient.

The apparatus 340 can further include electrodes/needles configured to deliver a PEF and/or agents to the target tissue in lieu of or in addition to monitoring the target tissue. For example, the apparatus can include additional electrodes or needles 350 that deliver the PEF and/or agents to the target tissue transvascularly. Alternatively, the electrodes 349b can be configured to deliver the PEF and/or agents transvascularly in addition to monitoring the tissue outside of the vessel.

With reference now to FIG. 14, an alternative embodiment of the extravascular PEF system of FIG. 4 is described comprising monitoring elements. In FIG. 14, electrode catheter 212 comprises bipolar PEF-delivery electrodes 214 and monitoring electrodes 216, which also may be used in a bipolar fashion. The monitoring electrodes and the PEF-delivery electrodes are electrically coupled to modified pulse generator 50′ by wires 211a and 211b, respectively. In use, PEF therapy is delivered via the PEF-delivery electrodes, and electroporation induced by the PEF therapy is monitored via the monitoring electrodes 216. The PEF therapy preferably is adjusted or controlled in response to the monitoring data received from electrodes 216. Modified pulse generator 50′ is configured to deliver the PEF therapy across the PEF-delivery electrodes and to deliver low voltage signals across the monitoring electrodes, as well as to collect and analyze the monitoring data collected with the monitoring electrodes.

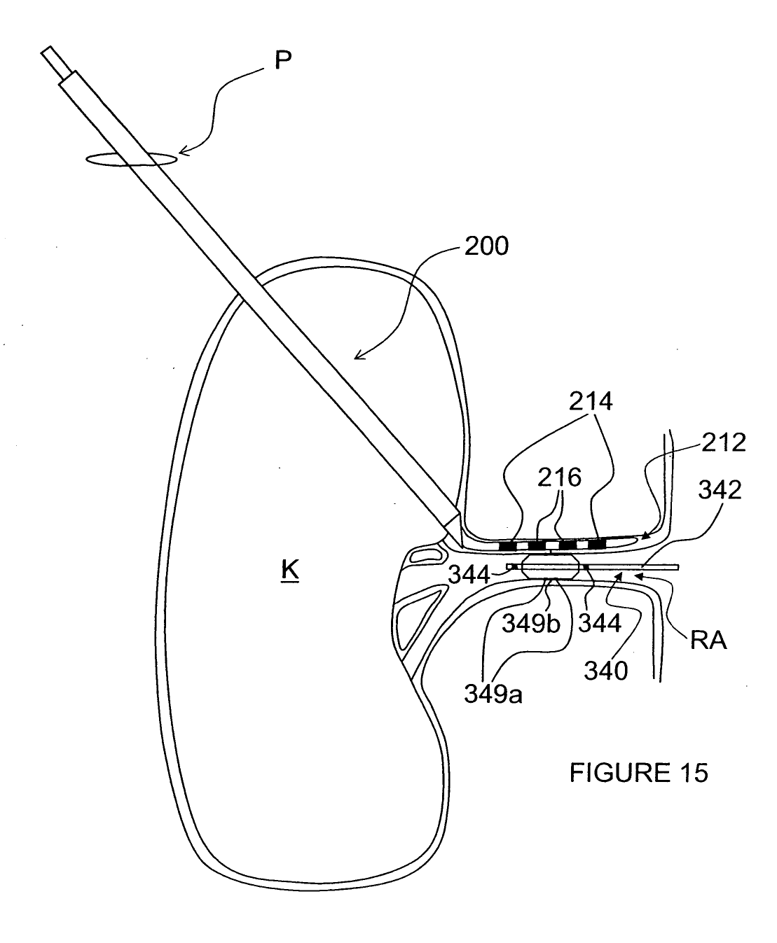

Referring now to FIG. 15 in conjunction with FIGS. 13 and 14, combination intravascular, transvascular and extravascular apparatus for inducing, monitoring and controlling PEF therapy is described. In FIG. 15, apparatus 200 of FIG. 14 has been positioned extravascularly, while a variation of apparatus 340 of FIG. 13 is positioned intravascularly and transvascularly. In FIG. 15, non-target tissue monitoring electrodes 349a of catheter 342 contact, but do not penetrate, the vessel wall, while target tissue monitoring electrodes 349b are positioned transvascularly within the adventitia.

The apparatus of FIG. 15 facilitates monitoring of both intravascular and extravascular non-target tissue, as well as adventitially-disposed target tissue. Specifically, monitoring electrodes 216 are positioned for monitoring of the external wall of the vessel, while monitoring electrodes 349a are positioned for monitoring of the internal wall of the vessel. Furthermore, monitoring electrodes 349b are transvascularly positioned for monitoring of target neural tissue within the adventitia. PEF therapy may be delivered intravascularly via PEF-delivery electrodes 344, extravascularly via bipolar electrodes 214, or a combination thereof.

Although FIG. 15 illustratively comprises combination apparatus having intravascular, extravascular and transvascular components, it should be understood that any desired subset of intra-, extra- and transvascular components may be utilized, as desired. Furthermore, although the transvascular components of the apparatus of FIG. 15 illustratively originate intravascularly, it should be understood that the components alternatively may originate extravascularly. Further still, although the apparatus of FIG. 15 illustratively is configured to deliver PEF therapy both intravascularly and extravascularly, it should be understood that the apparatus alternatively may be configured for delivering the therapy solely intravascularly or solely extravascularly. PEF therapy also may be delivered transvascularly. Additionally, PEF therapy may be delivered from within one vessel in the renal vasculature and monitored from within a different vessel in the renal vasculature. For example, PEF therapy may be delivered from electrodes positioned within or across a renal artery and monitored via electrodes positioned within or across a renal vein.

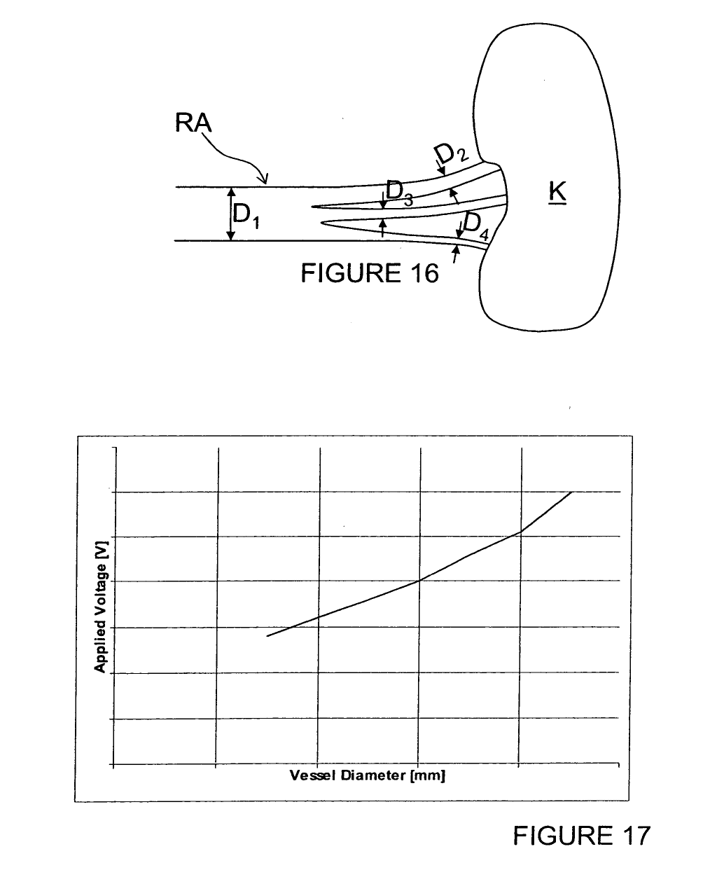

With reference now to FIGS. 16 and 17, an upward-sloping relationship between vessel diameter and required applied voltage necessary to achieve a desired field strength in target neural fibers that contribute to renal function from an intravascularly-delivered PEF therapy is described. In order to apply a relatively consistent field strength to neural fibers that contribute to renal function, it may be necessary to apply a PEF with greater voltage in larger vessels. This upward-sloping relationship between voltage and vessel size allows for customization of the pulsed electric field based on the vessel size to be treated. Customization may be performed for each individual patient based on his or her specific vessel size, may be performed based on an average vessel size for a given location within renal vasculature, may be performed based on a combination of these factors or on other factors.

As seen in FIG. 16, the renal vasculature may have a variety of branches requiring treatment (for the purposes of illustration, the vasculature comprises three distal branches; however, any alternative number of branches may be present). The main branch of the renal artery RA generally has a diameter D₁ that is larger than the diameters D₂, D₃ and D₄ of the distal branches. In FIG. 16, D₁>D₂>D₃>D₄, though the diameters may vary in a different manner, and/or a different number of branches may be present. The PEF system or the medical practitioner may determine these vessel sizes and modify the PEF therapy, as appropriate. Thus, when treating the patient of FIG. 16, voltage would be increased in the main branch of the renal artery having diameter D₁ and sequentially lowered in the distal branches having diameters D₂, D₃ and D₄.

For a known separation distance between the PEF-delivery electrodes, FIG. 17 schematically illustrates the upward-sloping relationship between internally-applied voltage and vessel diameter for a given expected field strength [V/cm] near the adventitia of the vessel. Once a desired adventitial field strength is selected and the vessel diameter is determined, the necessary applied voltage may be determined for the given electrode spacing. Optionally, a three-dimensional graph may be utilized that plots field strength, applied voltage and vessel diameter against one another. PEF-delivery electrode separation distance also may be plotted or examined against any or all of field strength, applied voltage and vessel diameter.

As an example, modeling indicates that, for a pair of bipolar PEF-delivery electrodes spaced 5 mm apart and centered within the vessel, in order to achieve field strength of 180 V/cm in the adventitia of a 6 mm vessel, an applied voltage of about 200V would be required, while the same field strength in a vessel 4 mm in diameter would require an applied voltage of about 160V. These values are provided only for the purposes of illustration and should in no way be construed as limiting.

Temporarily blocking blood flow between the intravascular PEF-delivery electrodes, e.g., via an inflatable balloon, may locally increase impedance relative to regular blood flow. This may preferentially direct PEF therapy delivered across the electrodes into or through the vessel wall. This, in turn, may reduce the voltage required to achieve a desired field strength in the adventitia in a vessel of a given diameter, relative to unimpeded blood flow.

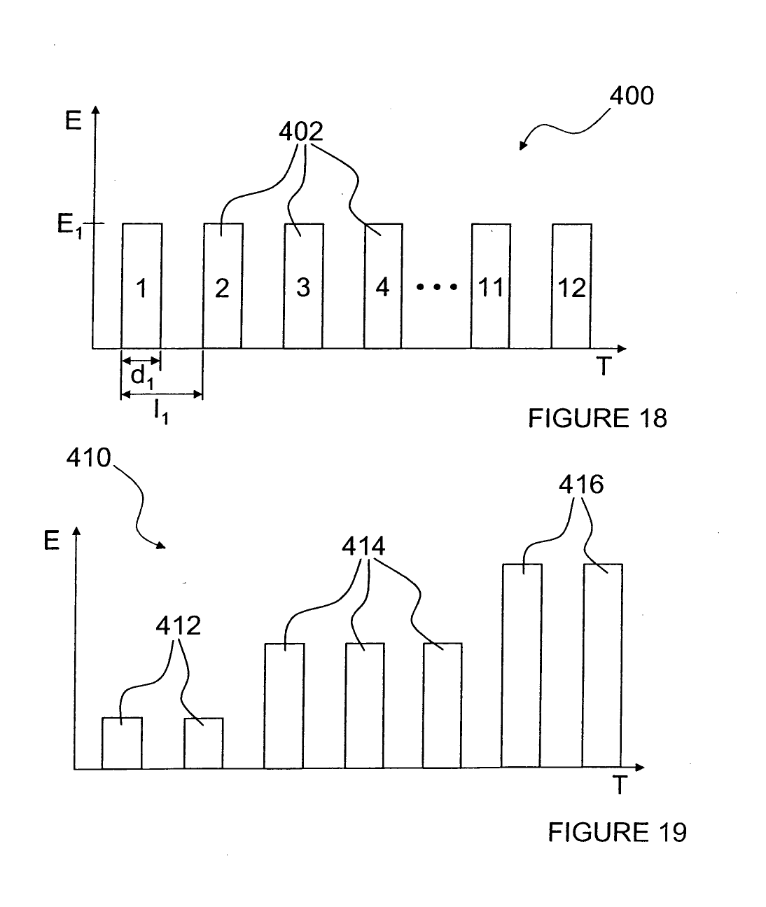

Referring now to FIGS. 18-27, illustrative PEF waveforms or pulse trains for inducing desired electroporative effects are described, such as in vivo, irreversible electroporation of nerves innervating the kidney. The PEF waveforms preferably do not electroporate or irreversibly electroporate non-target surrounding tissue, such as renal vasculature, kidney tissues, adrenal glands, lymph nodes, etc. These waveforms also may be applied in other in vivo applications wherein target tissue is more susceptible to electroporation than surrounding tissue. The waveforms, may, for example, be delivered via any of the previously described intravascular, extravascular or transvascular techniques.

PEF waveform 400 of FIG. 18 comprises a non-varying pulse train having one or more pulses 402 of equal voltage or equal field strength E₁, equal duration d₁ and equal interval I₁, delivered over time T. As an example, in one embodiment, waveform 400 might have a field strength of 150 V/cm, a pulse duration of 2 ms, an interval of 1 second, and 12 pulses in total, though any other parameters may be provided. This waveform may be repeated or modified as desired, for example, in response to monitoring data collected during or after delivery of the waveform. The interval between delivery of individual pulses and/or between delivery of subsequent waveforms may be used to deliver a low voltage signal across monitoring electrodes for monitoring the effects of the PEF therapy, e.g., to measure impedance or conductivity of target or non-target tissue using, for example, the same or different electrodes than were used for PEF therapy delivery. It should be understood that such time gating of monitoring may be utilized with any of the waveforms described hereinafter.

In vitro experimentation has shown that altering various aspects of a PEF waveform can improve cell viability or survival. However, for the purposes of the present invention, it may be desirable to cause irreversible electroporation and cell death in target tissue. Thus, opposite alterations to those known to protect cells may be applied.

Waveform 410 of FIG. 19 alters the field strength E [V/cm] in a manner that might increase irreversible electroporation. Waveform 410 begins with one or more relatively lower field strength pulses 412, followed by one or more relatively higher field strength pulses 414. Still higher field strength pulses 416 may be applied, etc. Lower field strength pulses 412 may be used to initiate electroporation in target neural tissue with little or no electroporation in non-target surrounding tissues. Once the electroporative effect is initiated in the target tissue, higher field strength pulses 414 and/or 416 expand or increase the number of pores in the target tissue, resulting in cell death. Furthermore, waveforms such as waveform 410 that begin with relatively smaller amplitude (i.e., voltage or field strength) might reduce a sensation of pain felt by the patient and/or may reduce muscle spasm.

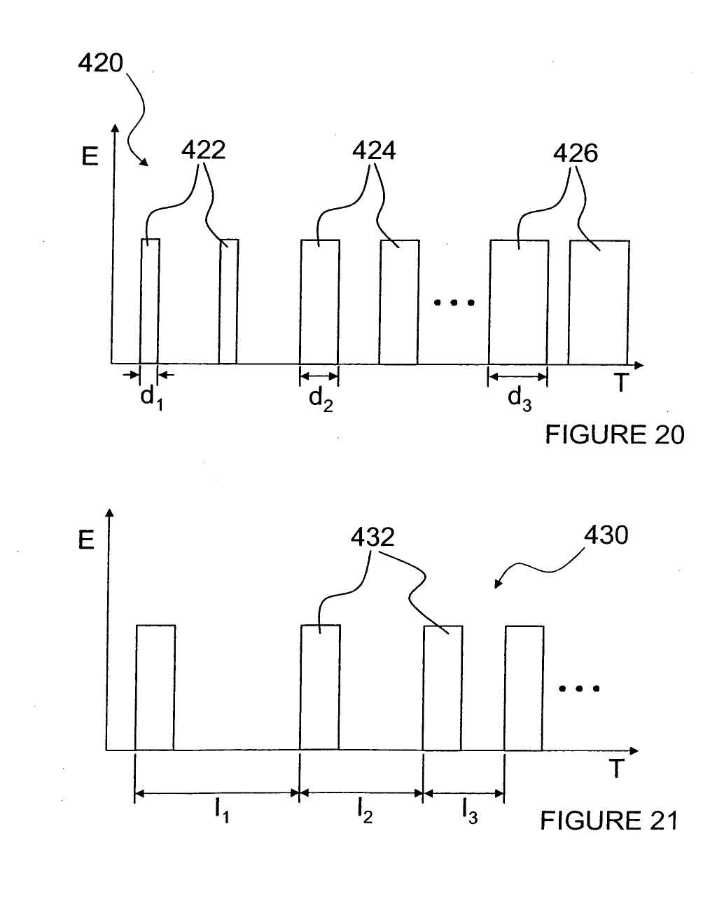

In FIG. 20, the pulse duration d of waveform 420 is ramped up or increased to enhance irreversible electroporation of the target tissue. Waveform 420 begins with one or more pulses 422 of relatively shorter duration d₁, followed by one or more pulses 424 of relatively longer duration d₂. The shorter duration pulses 422 may initiate electroporation in the target tissue with little or no electroporation in non-target surrounding tissues. The longer duration pulses 424 expand or increase the number of pores in the target tissue resulting in cell death. As will be apparent, still longer duration pulses, such as pulses 426 of duration d₃, may be provided as desired.

In FIG. 21, the time interval between pulses of waveform 430 is progressively decreased to enhance irreversible electroporation of the target tissue. Waveform 430 begins with interval I₁ between pulses 432. The interval is decreased to I₂, I₃, etc. It is known that electroporative pores close over time. By decreasing the time between each pulse, pores might expand or increase in number at a higher rate, potentially inducing irreversible electroporation with fewer total pulses.

A preferred pulse train for performing irreversible electroporation may involve a combination of variations in pulse amplitude or field strength, duration, and/or interval, as well as other parameters. In some embodiments, it may be desirable to alter multiple parameters within a single pulse to irreversibly electroporate target tissue while preferentially maintaining the viability of non-target tissue. Parameter variation optionally may be conducted manually or automatically in response to impedance or conductivity monitoring data obtained in the vicinity of the treatment site.

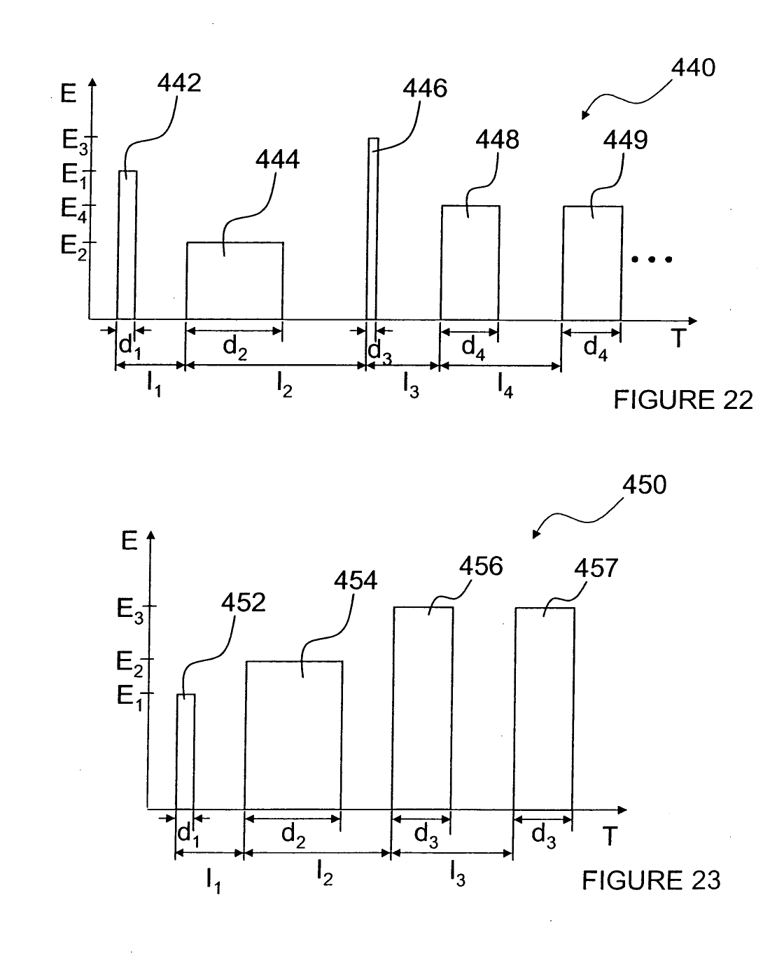

Waveform 440 of FIG. 22 provides an example of a waveform comprising variation along multiple parameters. Pulse 442 has field strength E₁, duration d₁ and interval I₁. Pulse 442 initiates pore formation in target tissue, such as renal nerves. Preferably, little, no or reduced electroporation is initiated in non-target tissue. Interval I₁ may be of a duration sufficient to preclude excessive heating of target or non-target tissue.

Pulse 444 of field strength E₂, duration d₂ and interval I₂, may be used to expand pores initiated by pulse 442. Although field strength E₂ is lower than field strength E₁, the longer duration d₂ may increase the total pore area and/or may generate heat in the target tissue, which may enhance the electroporative effect. Interval I₂ may be long enough to dissipate heat generated by pulse 444, or it may be short enough that some elevation in temperature persists upon application of pulse 446.

Pulse 446 of field strength E₃, which is larger than field strength E₂, may further increase pore area. The relatively shorter pulse duration d₃ may reduce heat generation as compared to pulse 444, and thus may require a relatively shorter interval I₃ to dissipate generated heat. Optional pulses 448 and 449 of reduced field strength E₄, increased duration d₄ and increased interval 14 relative to pulse 446 may further expand pores in target tissue, if needed, to achieve irreversible electroporation.

Additional or fewer pulses may be used, as needed. Furthermore, the parameters of the pulses may be varied, as needed. Variations in the number and/or form of the pulses of which waveform 440 is comprised may, for example, be determined in response to monitoring data collected in the vicinity of the treatment site.

With reference to FIG. 23, waveform 450 provides another example of a waveform comprising variation along multiple parameters. Pulse 452 comprises field strength E₁, duration d₁ and interval I₁. The pulse initiates electroporation in target tissue. The pulse interval is sufficient to preclude excessive heat generation in non-target tissue.

Pulse 454 is of larger field strength E₂ and longer pulse duration d₂ to increase pore surface area in target cell membranes. Interval I₂ may or may not equal interval I₁. Pulses 456 and 457, which irreversibly electroporate target tissue, are of larger field strength E₃ and of shorter pulse duration d₃ than the field strength and pulse duration of pulse 454.

The pulses of waveform 450 may induce electroporation in non-target tissue. However, if electroporation is induced in such non-target tissue, the pulse train preferable induces only reversible electroporation in the non-target tissue. Various protective measures may be employed to further protect or repair non-target tissues.

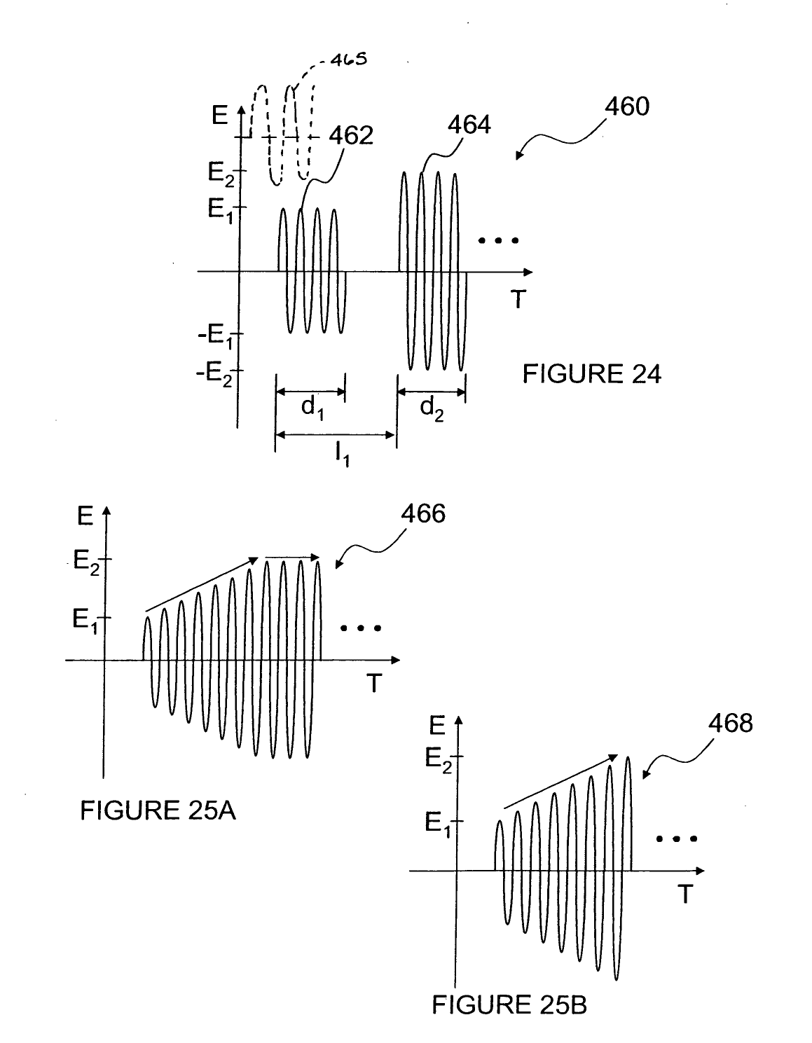

Referring now to FIG. 24, pulsed alternating current waveform 460 also may be utilized. The same alterations to pulse and pulse train parameters may be employed as in the previous DC embodiments to achieve a desired effect, such as alteration of pulse (peak) amplitude or field strength, duration, and/or interval. Additionally, pulse frequency may be altered in an AC waveform. Waveform 460 illustratively comprises AC pulse 462 of lower peak field strength magnitude E₁ than the peak field strength magnitude E₂ of AC pulse 464. This may also potentially be accomplished by DC-shifted AC waveforms as shown by waveform 465 (broken line) in FIG. 24.

In addition to alteration between pulses, parameter alteration also may be provided within a pulse. In FIG. 25A, pulse 466 comprises a ramp in peak field strength magnitude from initial peak field strength magnitude E₁, followed by a period of constant peak field strength magnitude E₂. Alternative pulse 468 of FIG. 25B comprises a continuous ramp in peak field strength magnitude from an initial magnitude E₁ to a final magnitude E₂.

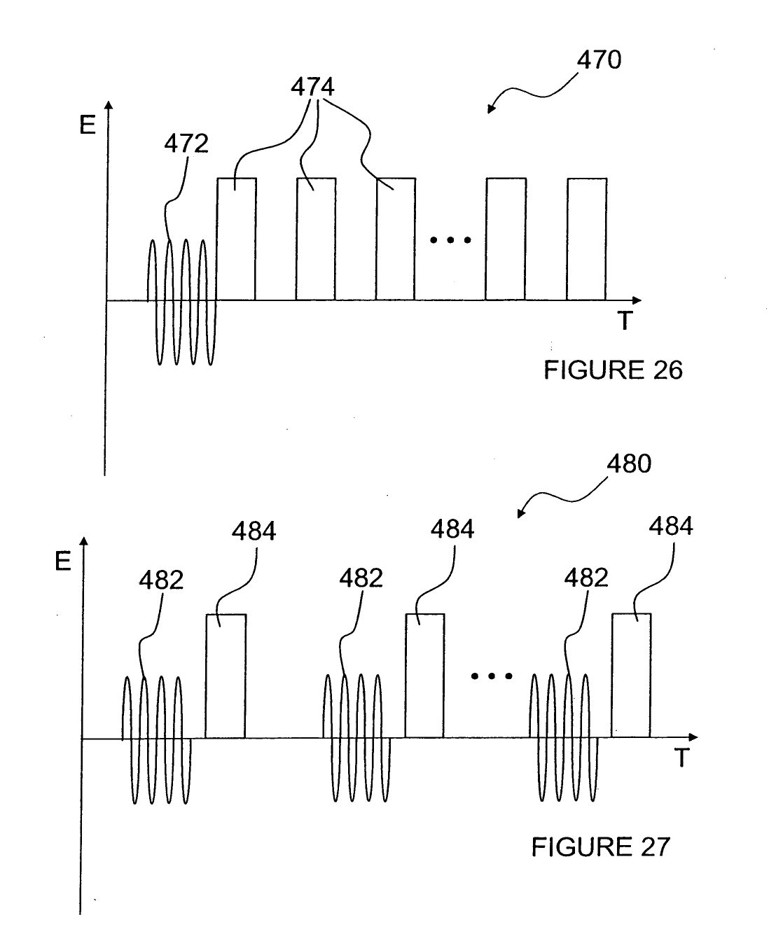

With reference to FIG. 26, it has been observed in animal studies that application of DC pulses can cause a muscular response wherein vessel spasm and skeletal muscle contraction can occur. It has also been observed that application of a 500 kHz radiofrequency alternating current substantially reduces vessel spasm and muscle contraction. It is expected that alternative AC frequencies would have a similar effect, and 500 kHz should in no way be construed as limiting.

While it may be desirable to use an RF current to reduce or eliminate spasm and muscle contraction, the literature suggests that AC waveforms provide less cell-size specificity. In the case of in vivo electroporation, cell-size specificity may be of significant utility when target cells are larger than non-target cells. FIG. 26 provides a combination AC and DC waveform that is expected to provide both cell-size specificity and reduction in spasm or muscle contraction. Waveform 470 comprises initial AC pulse 472 followed by a series of DC pulses 474. The initial AC pulse may attenuate or abolish adverse muscular responses, while the DC pulses may achieve desired cell-size selectivity.

The peak field strength and/or the duration of the AC pulse may be less than, equal to, or greater than the field strength and/or duration, respectively, of the DC pulses. Furthermore, the parameters of the DC pulses may vary. Preferably, the interval between the AC pulse and the DC pulses is relatively short or is non-existent, such that muscular tissue cannot recover prior to initiation of the DC pulses. Optionally, multiple AC pulses may be provided in combination with one or more DC pulses. Waveform 480 of FIG. 27 comprises multiple AC pulses 482 in combination with multiple DC pulses 484.

Any of the embodiments of the present invention described herein optionally may be configured for infusion of agents into the treatment area before, during or after energy application, for example, to create a working space to facilitate electrode placement, to enhance or modify the neurodestructive or neuromodulatory effect of applied energy, to protect or temporarily displace non-target cells, and/or to facilitate visualization. Additional applications for infused agents will be apparent. If desired, uptake of infused agents by cells may be enhanced via initiation of reversible electroporation in the cells in the presence of the infused agents. The infusate may comprise, for example, fluids (e.g., heated or chilled fluids), air, CO₂, saline, heparin or heparinized saline, hypertonic saline, contrast agents, gels, conductive materials, space-occupying materials (gas, solid or liquid), protective agents, such as Poloxamer-188, anti-proliferative agents, Sirolimus, or other drugs and/or drug delivery elements. Variations of the present invention additionally or alternatively may be configured for aspiration. Agent infusion or aspiration may be performed in response to monitoring data obtained in the vicinity of the treatment site.

Although preferred illustrative variations of the present invention are described above, it will be apparent to those skilled in the art that various changes and modifications may be made thereto without departing from the invention. For example, although the variations primarily have been described for use in combination with pulsed electric fields, it should be understood that any other electric field may be delivered as desired. It is intended in the appended claims to cover all such changes and modifications that fall within the true spirit and scope of the invention.

I/we claim:

1. Apparatus for inducing, monitoring and controlling renal neuromodulation, the apparatus comprising:
a pulsed electric field generator;
at least one electrode configured for placement proximate a neural fiber that contributes to renal function,
wherein the electrode is electrically coupled to the pulsed electric field generator for delivering a pulsed electric field to the neural fiber while the electrode is located proximate the neural fiber and/or is configured to monitor electroporation in tissue exposed to the pulsed electric field; and
a module operatively coupled to the electrode and configured to output data indicative of electroporation.

2. The apparatus of claim 1, wherein the electrode comprises at least one pulsed electric field-delivery electrode and at least one monitoring electrode.

3. The apparatus of claim 2, wherein the pulsed electric field-delivery electrode comprises a bipolar electrode pair.

4. The apparatus of claim 2, wherein the monitoring electrode comprises a pair of monitoring electrodes.

5. The apparatus of claim 1, wherein the electrode is configured to monitor electroporation in target tissue exposed to the pulsed electric field.

6. The apparatus of claim 5, wherein the electrode is configured to monitor electroporation induced in the neural fiber by the pulsed electric field.

7. The apparatus of claim 1, wherein the electrode is configured to monitor electroporation in non-target tissue exposed to the pulsed electric field.

8. The apparatus of claim 7, wherein the electrode is configured to monitor electroporation in wall tissue of renal vasculature.

9. The apparatus of claim 1, wherein the electrode is configured to monitor impedance, conductance or conductivity in tissue exposed to the pulsed electric field.

10. The apparatus of claim 1, wherein the electrode is configured for placement adjacent renal vasculature.

11. The apparatus of claim 10, wherein the electrode is configured for placement external to the renal vasculature.

12. The apparatus of claim 10, wherein the electrode is configured for placement within the renal vasculature.

13. The apparatus of claim 10, wherein the electrode is configured for placement across a wall of the renal vasculature.

14. The apparatus of claim 1 further comprising a feedback mechanism for altering delivery of the pulsed electric field in response to electroporation monitoring data collected with the electrode.

15. The apparatus of claim 14, wherein the feedback mechanism is configured to halt delivery of the pulsed electric field in response to monitoring data indicative of undesirable electroporation.

16. The apparatus of claim 14, wherein the feedback mechanism is configured to vary at least one parameter of the pulsed electric field in response to the monitoring data.

17. The apparatus of claim 16, wherein the parameter is chosen from the group consisting of voltage, field strength, pulse width, pulse duration, pulse shape, pulse interval, duty cycle, number of pulses, and combinations thereof.

18. The apparatus of claim 1, wherein the apparatus is configured to orient a longitudinal portion of the pulsed electric field with a longitudinal dimension of the neural fiber that contributes to renal function.

19. The apparatus of claim 1, wherein the pulsed electric field generator is configured to produce a pulsed electric field that induces irreversible electroporation in the neural fiber that contributes to renal function.

20. A method for inducing, monitoring and controlling renal neuromodulation, the method comprising:
positioning at least one electrode proximate to a neural fiber that contributes to renal function of a patient;
delivering a pulsed electric field to modulate the neural fiber; and
monitoring electroporation via the electrode in tissue exposed to the pulsed electric field.

21. The method of claim 20, wherein positioning the electrode further comprises positioning at least one pulsed electric field-delivery electrode and at least one monitoring electrode;
wherein delivering the pulsed electric field further comprises delivering the pulsed electric field via the pulsed electric field-delivery electrode; and
wherein monitoring electroporation further comprising monitoring electroporation via the monitoring electrode.

22. The method of claim 21, wherein delivering the pulsed electric field further comprises delivering the pulsed electric field across a bipolar electrode pair.

23. The method of claim 21, wherein monitoring electroporation further comprises monitoring electroporation across a pair of monitoring electrodes.

24. The method of claim 20, wherein monitoring electroporation further comprises monitoring electroporation in target tissue exposed to the pulsed electric field.

25. The method of claim 20, wherein monitoring electroporation further comprises monitoring electroporation induced in the neural fiber by the pulsed electric field.

26. The method of claim 20, wherein monitoring electroporation further comprises monitoring electroporation in non-target tissue exposed to the pulsed electric field.

27. The method of claim 26, wherein monitoring electroporation further comprises monitoring wall tissue of renal vasculature.

28. The method of claim 20, wherein monitoring electroporation further comprises monitoring impedance, conductance or conductivity in tissue exposed to the pulsed electric field.

29. The method of claim 20, wherein positioning the electrode proximate to the neural fiber further comprises positioning the electrode adjacent renal vasculature.

30. The method of claim 29, wherein positioning the electrode adjacent renal vasculature further comprises positioning the electrode external to the renal vasculature.

31. The method of claim 29, wherein positioning the electrode adjacent renal vasculature further comprises positioning the electrode within the renal vasculature.

32. The method of claim 29, wherein positioning the electrode adjacent renal vasculature further comprises positioning the electrode across a wall of the renal vasculature.

33. The method of claim 20 further comprising altering delivery of the pulsed electric field in response to monitoring data.

34. The method of claim 33, wherein altering delivery of the pulsed electric field further comprises halting delivery of the pulsed electric field in response to monitoring data indicative of undesirable electroporation.

35. The method of claim 34, wherein undesirable electroporation comprises irreversible electroporation of non-target tissue.

36. The method of claim 33, wherein altering delivery of the pulsed electric field further comprises varying at least one parameter of the pulsed electric field in response to the monitoring data.

37. The method of claim 20, wherein delivering the pulsed electric field further comprises orienting the pulsed electric field with a longitudinal dimension of the neural fiber that contributes to renal function.

38. The method of claim 20, wherein delivering the pulsed electric field to modulate the neural fiber further comprises inducing irreversible electroporation in the neural fiber.

39. The method of claim 20, wherein delivering the pulsed electric field to modulate the neural fiber further comprises denervating the neural fiber.

40. The method of claim 20 further comprising infusing an agent to protect or repair non-target cells from effects of the pulsed electric field.

41. The method of claim 40 wherein infusing the agent further comprises infusing the agent in response to monitoring data indicative of undesirable electroporation of non-target cells.

42. The method of claim 20 further comprising infusing an agent to alter the susceptibility of cells to electroporation.

43. The method of claim 20 further comprising protecting or repairing non-target cells from effects of the pulsed electric field.

44. The method of claim 31 further comprising, prior to delivering the pulsed electric field, monitoring impedance, conductance or conductivity within the vasculature via the electrode in order to determine whether the electrode is positioned in an adequately-sized vessel for delivery of the pulsed electric field.

45. The method of claim 31 further comprising monitoring patency of the renal vasculature via the electrode.

46. The method of claim 20, wherein delivering the pulsed electric field via the electrode further comprises multiplexing delivery of the pulsed electric field between a plurality of pulsed electric field-delivery electrodes.

47. The method of claim 20, wherein monitoring electroporation via the electrode further comprises multiplexing monitoring between a plurality of monitoring electrodes.

48. The method of claim 20, wherein monitoring electroporation via the electrode further comprises delivering a low voltage signal across tissue exposed to the pulsed electric field, and monitoring a response of the tissue to the low voltage signal.

49. The method of claim 20, wherein delivering the pulsed electric field and monitoring electroporation further comprises both delivering and monitoring via the at least one electrode.

50. The apparatus of claim 1, wherein the electrode is both electrically coupled to the pulsed electric field generator for delivering a pulsed electric field to the neural fiber while the electrode is located proximate the neural fiber and configured to monitor electroporation in tissue exposed to the pulsed electric field.

51. Apparatus for inducing, monitoring and controlling renal neuromodulation, the apparatus comprising:
an electrode configured for placement proximate a neural fiber that contributes to renal function, the at least one electrode configured to deliver a pulsed electric field to the neural fiber and/or to monitor electroporation in tissue exposed to the pulsed electric field; and
a feedback mechanism for altering delivery of the pulsed electric field in response to electroporation monitoring signals sensed by the electrode.

52. A method for inducing, monitoring and controlling renal neuromodulation, the method comprising:
positioning at least one electrode proximate to a neural fiber that contributes to renal function of a patient;
delivering a pulsed electric field via the electrode to modulate the neural fiber;
monitoring electroporation in tissue exposed to the pulsed electric field; and
altering delivery of the pulsed electric field in response to monitoring data.
